(19) United States
(12) Patent Application Publication
(10) Pub. No.: US 2025/0361543 A1
(43) Pub. Date: Nov. 27, 2025

(54) Instrumentation for Rapid Antimicrobial Susceptibility Testing from Bodily Fluids and Cultures

(71) Applicant: Georgia Tech Research Corporation, Atlanta, GA (US)

(72) Inventors: Robert Martin Dickson, Atlanta, GA (US); Prakash C. Khanal, Atlanta, GA (US)

(21) Appl. No.: 18/874,612

(22) PCT Filed: Jun. 15, 2023
(86) PCT No.: PCT/US2023/068517

§ 371 Date: Dec. 13, 2024

Related U.S. Application Data
(60) Provisional application No. 63/366,422, filed on Jun. 15, 2022.

(51) Int. Cl. C12Q 1/18; G01N 21/25

(52) U.S. Cl. CPC C12Q 1/18; G01N 21/253; G01N 2201/062

(57) ABSTRACT

An exemplary embodiment of the present disclosure provides a system for determining a minimum inhibitory concentration of an antimicrobial agent. The system can include a plurality of containers, a light source disposed on a first side of the plurality of containers and configured to shine a light through the plurality of containers, a detector, one or more processors, and a memory storing instructions thereon that, when executed by the one or more processors, cause the one or more processors to capture, with the detector, an intensity profile of at least one of the plurality of containers, determine, from the intensity profile, a first intensity of the light at a first wavelength range, and compare the first intensity of the light to a control.

### CROSS-REFERENCE TO RELATED APPLICATIONS

This application claims the benefit of U.S. Provisional Application Ser. No. 63/366,422, filed on 15 Jun. 2022, which is incorporated herein by reference in its entirety as if fully set forth below.

### FIELD OF THE DISCLOSURE

The various embodiments of the present disclosure relate generally to systems and methods for testing for antimicrobial resistance, and more particularly to rapid antibiotic susceptibility testing directly from bodily fluids and cultures.

### BACKGROUND

Despite the wide availability of antibiotics, bacterial infections remain a major cause of mortality and morbidity as they rapidly become resistant to even the most potent drugs. The inability to rapidly diagnose infection-causing bacteria and determine their antimicrobial susceptibilities poses serious diagnostic limitations, while simultaneously increasing antimicrobial resistance (AMR). Especially true for patients with blood stream infections (BSIs), the >60-hr time-to-result for BSI diagnosis is at odds with the only known indicator of sepsis survival-time elapsed before initiation of proper antibiotics treatment. Although new molecular approaches promise to speed bacterial species identification to within a few hours of positive blood culture, the time-consuming blood culture and subsequent delay in antimicrobial susceptibility testing (AST) mutes these advances. Additionally, the complexity and cost of both identification and susceptibility determinations is prohibitive for all but high-resource hospital labs. While both identification and susceptibility profile are considered actionable treatment information, accelerating susceptibility determinations after positive blood culture would remove the greatest impediment to rapid, appropriate treatment. Further, decreasing cost and sample handling for both identification and ASTs would drastically improve patient outcomes and decrease incidence of AMR in both high- and low-resource settings. This rapid, informed approach would also decrease the overuse of inappropriate empiric antibiotics that contributes to the alarming rise in antibiotic resistance.

Adults exhibiting sepsis (an immune response to bacteremia caused by 1˜100 colony-forming-units (CFU)/mL blood) represent an extreme challenge for fast treatment. The low CFU densities thus require ˜24-hr blood culture-based amplification as the necessary first step in any BSI treatment guidance. While identification of resistance genetic markers holds promise for shortening time to antibiotic susceptibility determination, only a subset of resistance genes have been clearly defined. Thus, a phenotypic approach is needed for catching bacteria with inducible resistances that contribute to treatment failures and populations with heterogeneous resistance profiles. Although the need for rapid antibiotic susceptibility information has motivated efforts to reduce AST time, no rapid ASTs have yet demonstrated themselves sufficiently general and accurate for adoption in clinical applications. For example, automated bacteria identification and ASTs with a mean time-to-result of 9 hour (post blood culture) have been developed based on turbidimetric and fluorescence end point measurements; however, these methods were incapable of detecting β-lactam susceptibility. Growth kinetics monitoring enabled 6-19-hour ASTs, but only for a few fast-growing gram-negative species. Bioluminescent AST assays give good agreement with standard methods, but the sensitivity relies on ATP detection and suffers from substrate instability, causing spurious results. In all cases, additional complications arise from inherent bacterial population heterogeneity in response to antibiotic treatment.

Therefore, there is a need for a fast, simple and easy to use antibiotic susceptibility tests and methods to test for antimicrobial resistant infections using bodily fluids and cultures.

### BRIEF SUMMARY

The present disclosure relates to systems and methods for detecting antimicrobial resistance in a sample. An exemplary embodiment of the present disclosure provides a system for determining a minimum inhibitory concentration (MIC) of an antimicrobial agent. The system can include a plurality of containers, a light source disposed on a first side of the plurality of containers and configured to shine a light onto the plurality of containers, a detector, one or more processors, and a memory storing instructions thereon that, when executed by the one or more processors, cause the one or more processors to capture, with the detector, an intensity profile of at least one of the plurality of containers, determine, from the intensity profile, a first intensity of the light at a first wavelength range, and compare the first intensity of the light to a control. Each container can be configured to contain at least a portion of a biological sample and an antimicrobial agent.

In any of the embodiments disclosed herein, each of the plurality of containers can be configured to contain varying concentrations of the antimicrobial agent, and comparing the first intensity to the control can be indicative of an effectiveness of the concentration of the antimicrobial agent in the respective container.

In any of the embodiments disclosed herein, the instructions can further cause the one or more processors to determine, from the intensity profile, a second intensity of the light at a second wavelength range and compare the second intensity of the light to the control.

In any of the embodiments disclosed herein, the instructions can further cause the one or more processors to determine, from the intensity profile, a third intensity of the light at a third wavelength range and compare the third intensity of the light to the control.

In any of the embodiments disclosed herein, the light source can further include three constituent components at the first, second, and third wavelength ranges.

In any of the embodiments disclosed herein, the first, second, and third wavelength ranges corresponding to red, green, and blue light wavelength ranges respectively.

In any of the embodiments disclosed herein, the detector can be one of a plurality of detectors, each detector of the plurality of detectors aligned with a respective container of the plurality of containers.

In any of the embodiments disclosed herein, the detector can include a camera, and the intensity profile can be determined based at least in part on an image captured by the camera.

In any of the embodiments disclosed herein, the light source can include a plurality of light emitting diodes (LEDs), each LED of the plurality of LEDs being aligned with a respective container of the plurality of containers and opposite a respective detector.

In any of the of the embodiments disclosed herein, the detector and the light source can be disposed on the first side of the plurality of containers, and the intensity profile can be based on light reflected from the plurality of containers.

In any of the embodiments disclosed herein, an incubator configured to contain the plurality of containers.

In any of the embodiments disclosed herein, the system can further include a transparent cover film covering the plurality of containers, and the instructions further cause the one or more processors to maintain a temperature gradient in the incubator such that condensation does not form on the transparent cover film.

In any of the embodiments disclosed herein, the plurality of containers can include a well plate.

In any of the embodiments disclosed herein, positive and/or negative controls can be disposed in a portion of the plurality of containers.

Another exemplary embodiment of the present disclosure provides a method for determining a minimum inhibitory concentration of an antimicrobial agent. The method can include combining a biological sample with varying concentrations of an antimicrobial agent in a plurality of containers, incubating the plurality of containers, exposing the plurality of containers to a light, capturing an intensity profile of the plurality of containers, determining, from the intensity profile, a first intensity of the light at a first wavelength range for each container of the plurality of containers, comparing the first intensity to a control, and determining, based on the comparison, an effectiveness of the antimicrobial agent for each of the varying concentrations.

In any of the embodiments disclosed herein, capturing the intensity profile can include detecting light reflected from the plurality of containers.

In any of the embodiments disclosed herein, capturing the intensity profile can include detecting light projected through the plurality of containers.

In any of the embodiments disclosed herein, the method can further include determining, from the intensity profile, a second intensity of the light at a second wavelength range for each container of the plurality of containers and comparing the second intensity to the control.

In any of the embodiments disclosed herein, the method can further include determining, from the intensity profile, a third intensity of the light at a third wavelength range for each container of the plurality of containers and comparing the third intensity to the control.

In any of the embodiments disclosed herein, the method can further include sealing the plurality of containers, and incubating the plurality can further include maintaining a temperature gradient that prevents condensation from forming proximate the plurality of containers.

Another exemplary embodiment of the present disclosure provides a system for testing antimicrobial susceptibility. The system can include an incubator, a light source disposed in the incubator and configured to shine a light through a well plate including a plurality of wells, and a detector configured to capture an intensity profile of the well plate disposed between the detector and the light source. The well plate can contain, in the plurality of wells, biological samples and varying concentrations of an antimicrobial agent. The light can include three distinct subcomponents. The three distinct subcomponents can be different colors or different temperatures of white light.

In any of the embodiments disclosed herein, the detector can be one of a plurality of detectors, each detector of the plurality of detectors aligned with a respective well of the plurality of wells.

In any of the embodiments disclosed herein, the light source can include a plurality of light emitting diodes (LEDs). Each LED of the plurality of LEDs can be aligned with a respective container of the plurality of containers and opposite a respective detector and configured to emit light at three distinct wavelength ranges. The plurality of detectors can be configured to detect a respective intensity of each of the three wavelength ranges, the intensity indicative of an effectiveness of the respective concentration of the antimicrobial agent. Each of the plurality of detectors can capture an intensity profile from its respective container of the plurality of containers concurrently with other detectors capturing an intensity profile from their respective container, a portion of the detectors of the plurality detectors can capture their respective intensity profile at a time different from the other detectors, or each detector can capture its respective intensity profile sequentially.

In any of the embodiments disclosed herein, the system can further include a transparent cover film covering the well plate.

In any of the embodiments disclosed herein, the incubator can be configured to maintain a temperature gradient maintain a temperature gradient in the incubator such that condensation does not form on the transparent cover film.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides conventional susceptibility testing methods.

FIG. 2A provides a perspective view of a system for determining a minimum inhibitory concentration of an antimicrobial agent, in accordance with an exemplary embodiment of the present invention.

FIG. 2B provides a perspective view of a detector, a container, and a light source, in accordance with an exemplary embodiment of the present invention.

FIG. 3 provides a sample-antibiotics distribution scheme, in accordance with an exemplary embodiment of the present invention.

FIG. 4 illustrates a well plate containing portions of a biological sample at various time intervals, in accordance with an exemplary embodiment of the present invention.

FIG. 5 provides a flowchart for a method for determining a minimum inhibitory concentration of an antimicrobial agent, in accordance with an exemplary embodiment of the present invention.

FIG. 6 shows types and number of various types of Gram-negative samples assayed directly from positive blood cultures, in accordance with an exemplary embodiment of the present invention.

FIG. 7A provides a plot showing categorical agreement and error rates of plate reader assays, in accordance with an exemplary embodiment of the present invention.

FIG. 7B provides a plot showing categorical agreement and error rates of a three-wavelength range channel assay, in accordance with an exemplary embodiment of the present invention.

FIG. 8A provides a plot showing average essential agreement for various antibiotics in a plate reader assay, in accordance with an exemplary embodiment of the present invention.

FIG. 8B provides a plot showing average essential agreement for various antibiotics in a three-wavelength range channel assay, in accordance with an exemplary embodiment of the present invention.

FIG. 9A provides a plot showing categorical agreement of a plate reader, in accordance with an exemplary embodiment of the present invention.

FIG. 9B provides a plot showing categorical agreement of a three-wavelength range channel assay, in accordance with an exemplary embodiment of the present invention.

FIG. 9C provides a plot showing average essential agreement for various antibiotics in a plate reader assay, in accordance with an exemplary embodiment of the present invention.

FIG. 9D provides a plot showing average essential agreement for various antibiotics in a three-wavelength range channel assay, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

Though the terms “bacteria”, “bacterium”, and “bacterial” are used herein, the present disclosure can also be applied to other microorganisms such as fungi and others.

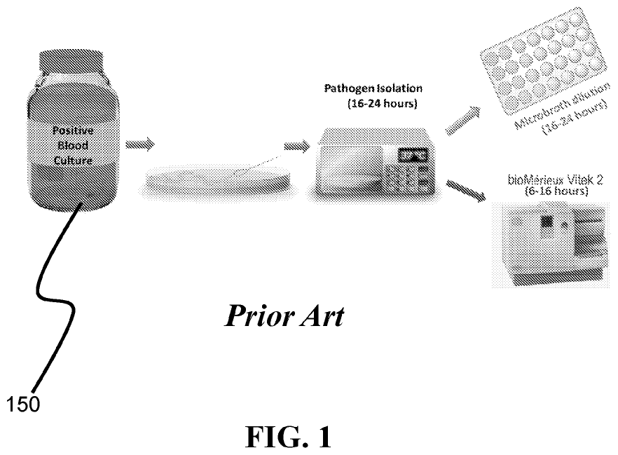

FIG. 1 provides conventional susceptibility testing methods.

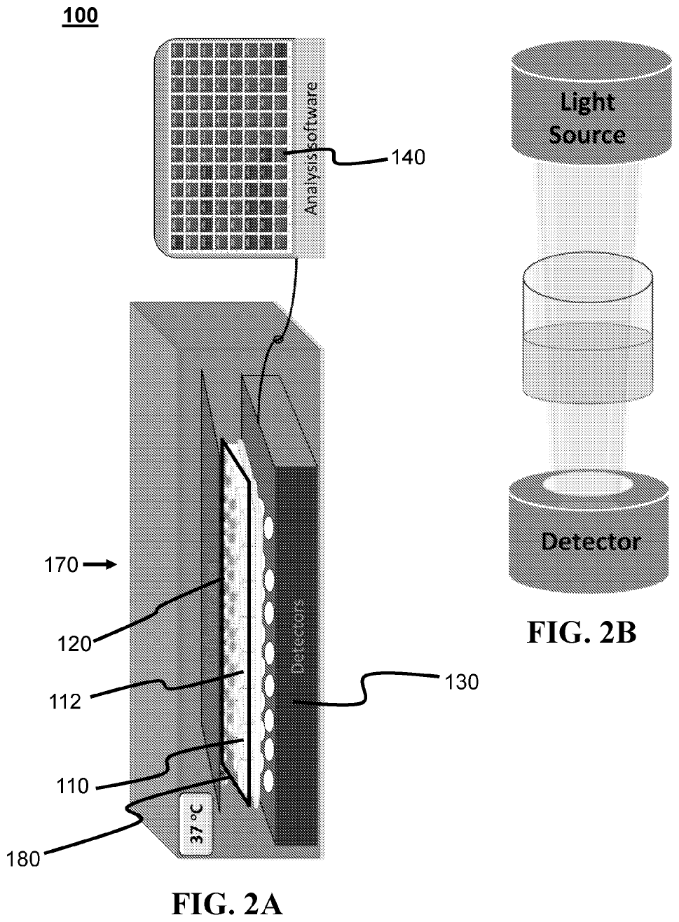

FIGS. 2A-2B provides a system 100 for determining a minimum inhibitory concentration of an antimicrobial agent 160. The system 100 can include a plurality of containers 110, a light source 120 disposed on a first side 112 of the plurality of containers 110 and configured to shine a light through the plurality of containers 110, a detector 130, one or more processors 140, and a memory storing instructions thereon that, when executed by the one or more processors 140, cause the one or more processors 140 to capture, with the detector 130, an intensity profile of at least one of the plurality of containers 110, determine, from the intensity profile, a first intensity of the light at a first wavelength range, and compare the first intensity of the light to a control. Each container can be configured to contain at least a portion of a biological sample 150 and an antimicrobial agent 160.

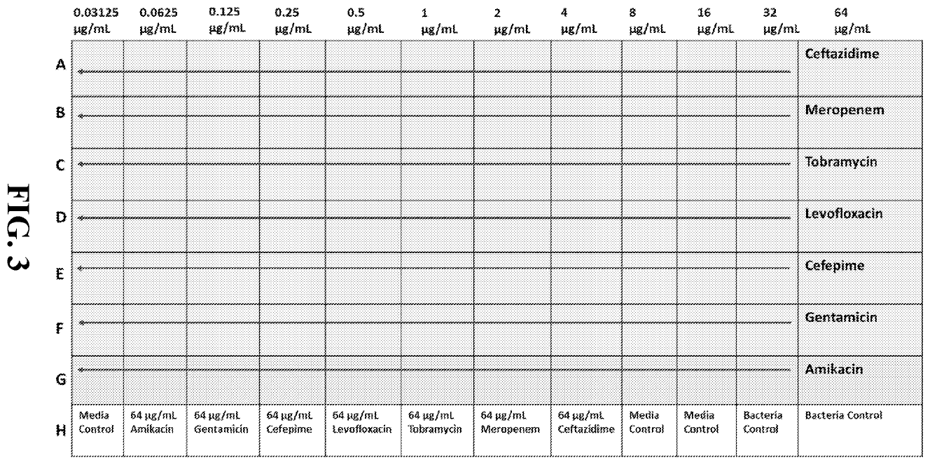

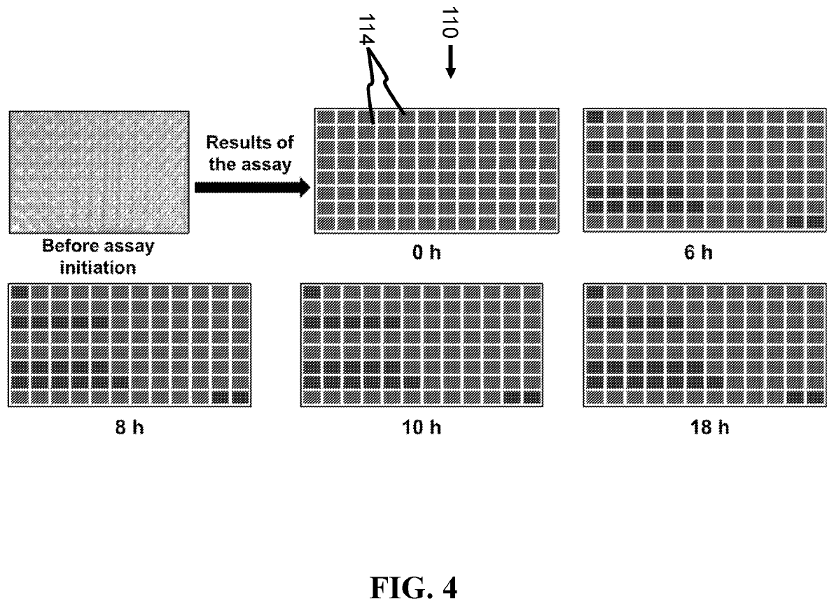

FIGS. 3-4 shows the plurality of containers 110 as a 96 well plate. As shown in FIG. 3, each of the plurality of containers 110 can be configured to contain varying concentrations of the antimicrobial agent 160. Comparing the first intensity to the control can be indicative of an effectiveness of the concentration of the antimicrobial agent 160 in the respective container. FIG. 3 shows concentrations of the seven indicated antimicrobial agents 160 doubling from one column to another from the top to the bottom of the well plate 110.

In any of the embodiments disclosed herein, the instructions can further cause the one or more processors 140 to determine, from the intensity profile, a second intensity of the light at a second wavelength range and compare the second intensity of the light to the control.

In any of the embodiments disclosed herein, the instructions can further cause the one or more processors 140 to determine, from the intensity profile, a third intensity of the light at a third wavelength range and compare the third intensity of the light to the control.

In any of the embodiments disclosed herein, the light source 120 can further include three constituent components at the first, second, and third wavelength ranges. In any of the embodiments disclosed herein, the first, second, and third wavelength ranges corresponding to red, green, and blue light wavelength ranges, respectively. These wavelength ranges can comprise the three red-green-blue (RGB) constituent components of white light.

In any of the embodiments disclosed herein, the detector 130 can be one of a plurality of detectors 130, each detector 130 of the plurality of detectors 130 aligned with a respective container of the plurality of containers 110.

In any of the embodiments disclosed herein, the detector 130 can include a camera, and the intensity profile can be determined based at least in part on an image captured by the camera.

In any of the embodiments disclosed herein, the light source 120 can include a plurality of light emitting diodes (LEDs), each LED of the plurality of LEDs being aligned with a respective container of the plurality of containers 110 and opposite a respective detector 130.

In any of the embodiments disclosed herein, an incubator 170 configured to contain the plurality of containers 110.

In any of the embodiments disclosed herein, the light source 120 can include a filter-based white light system. Thus a wavelength range is more general and would seem to encompass a non LED light source that is broadband but uses filters or monochrometer-based filtering of wavelengths for selective illumination

In any of the embodiments disclosed herein, the system 100 can further include a transparent cover film 180 covering the plurality of containers 110, and the instructions further cause the one or more processors 140 to maintain a temperature gradient in the incubator 170 such that condensation does not form on the transparent cover film 180.

In any of the embodiments disclosed herein, the plurality of containers 110 can include a well plate.

Stated otherwise, referring back to FIG. 2A-2B, the present disclosure provides a system 100 for testing antimicrobial susceptibility. The system 100 can include an incubator, a light source 120 disposed in the incubator and configured to shine a light through a well plate including a plurality of wells 114, and a detector 130 configured to capture an intensity profile of the well plate disposed between the detector 130 and the light source 120. The well plate can contain, in the plurality of wells 114, biological samples 150 and varying concentrations of an antimicrobial agent. The light can include three distinct subcomponents.

In any of the embodiments disclosed herein, the detector 130 can be one of a plurality of detectors 130, each detector 130 of the plurality of detectors 130 aligned with a respective well of the plurality of wells 114.

In any of the embodiments disclosed herein, the light source 120 can include a plurality of light emitting diodes (LEDs). Each LED of the plurality of LEDs can be aligned with a respective container of the plurality of containers 110 and opposite a respective detector 130 and configured to emit light at three distinct wavelength ranges. The plurality of detectors 130 can be configured to detect a respective intensity of each of the three wavelength ranges, the intensity indicative of an effectiveness of the respective concentration of the antimicrobial agent.

In any of the embodiments disclosed herein, the system 100 can further include a transparent cover film covering the well plate.

In any of the embodiments disclosed herein, the incubator can be configured to maintain a temperature gradient maintain a temperature gradient in the incubator such that condensation does not form on the transparent cover film.

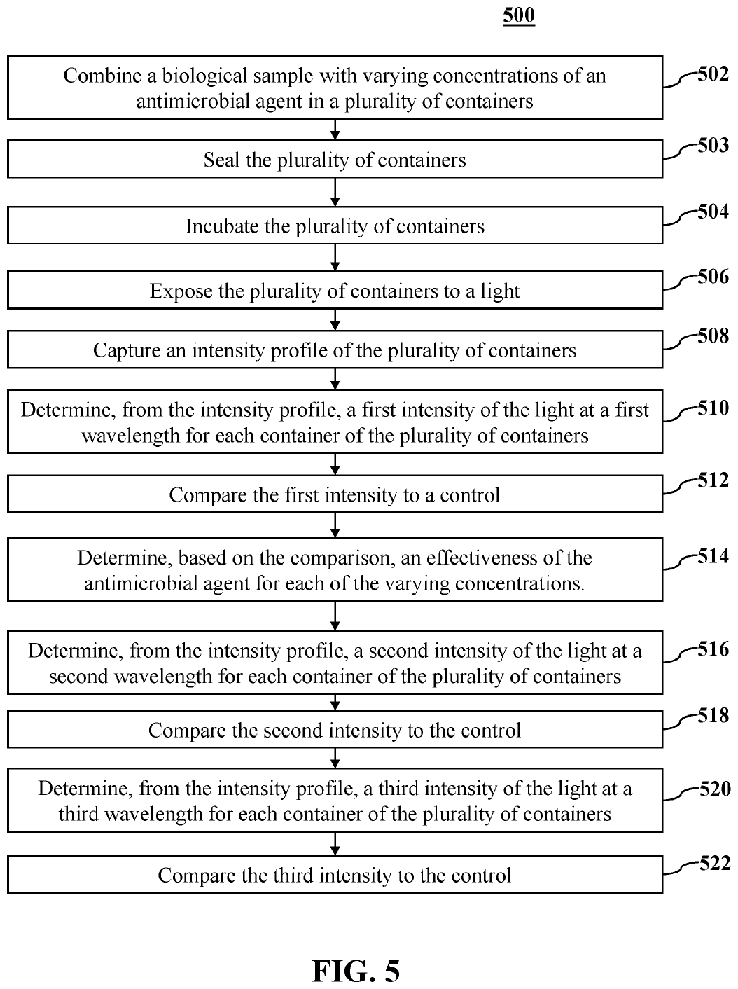

FIG. 5 provides a method 500 for determining a minimum inhibitory concentration of an antimicrobial agent. The method 500 can include at step 502 combining a biological sample with varying concentrations of an antimicrobial agent in a plurality of containers, at step 504 incubating the plurality of containers, at step 506 exposing the plurality of containers to a light, at step 508 capturing an intensity profile of the plurality of containers, at step 510 determining, from the intensity profile, a first intensity of the light at a first wavelength range for each container of the plurality of containers, at step 512 comparing the first intensity to a control, and at step 514 determining, based on the comparison, an effectiveness of the antimicrobial agent for each of the varying concentrations.

In any of the embodiments disclosed herein, the method 500 can further include at step 516 determining, from the intensity profile, a second intensity of the light at a second wavelength range for each container of the plurality of containers and at step 518 comparing the second intensity to the control.

In any of the embodiments disclosed herein, the method 500 can further include at step 520 determining, from the intensity profile, a third intensity of the light at a third wavelength range for each container of the plurality of containers and at step 522 comparing the third intensity to the control.

In any of the embodiments disclosed herein, the method 500 can further include at step 503 sealing the plurality of containers, and incubating the plurality can further include maintaining a temperature gradient that prevents condensation from forming proximate the plurality of containers. Preventing condensation from forming proximate the plurality of containers increases the fidelity with which the first, second, and third intensities of the light can be determined from the intensity profile. Method 500 can in some embodiments be carried out with system 100 and the subcomponents thereof as described herein.

The following examples further illustrate aspects of the present disclosure. However, they are in no way a limitation of the teachings or disclosure of the present disclosure as set forth herein.

### EXAMPLES

In some embodiments, the tests described herein demonstrates a fast, simple, and easy to use test that can outperform tests on the market. The tests can also provide a low labor cost. Disclosed in this example are systems and methods for performing ASTs directly from positive blood cultures. De-identified hospital patient positive blood cultures were received within 8 hours of being flagged positive. ASTs were performed using various antimicrobial agents, namely seven antibiotics (ceftazidime, meropenem, tobramycin, levofloxacin, cefepime, gentamicin, and amikacin). The positive blood cultures were diluted 250-fold in cation-adjusted Mueller-Hinton Broth (CAMHB) media and 100 μL of this diluted sample was dispensed into each well of a 96-well microtiter plate, already containing 100 μL of varying concentration of one antibiotic. This resulted in 500-fold final dilution of positive blood culture, and final antibiotic concentration in the range of 0.03125 μg/mL to 64 μg/mL. The sample-filled 96-well plate was sealed by sterile plastic film and incubated in a microtiter plate reader at 37° C. A temperature gradient of 0.5° C. was selected to prevent condensation on cover film. Each well was scanned every one hour from 400 to 895 nm with a 5 nm step starting from the time of incubation (0 hours) resulting in a total of nineteen spectra per well.

The as-received positive-blood cultures were diluted and plated on LB agar and incubated overnight at 37° C. A single colony from the LB agar plate was used as the inoculum in CAMHB media for ˜3 h at 37° C. in a shaker at ˜225 rpm followed by OD600 adjustment to ˜0.002. 100 μL of this bacterial suspension was dispensed in prefilled antibiotic-containing wells, resulting in final antibiotic concentration from 0.03125 μg/mL to 64 μg/mL. The 96 well plates were designed like that of plate reader assays. The sample dispensed 96-well plate were incubated at 37° C. for 18 h followed by MIC determination by visual inspection of turbidity. The MIC of each antibiotic for BMD was defined as the well with lowest concentration where there was absence of visual turbidity.

Relating to data analysis-spectra (400 to 895 nm) vs time were acquired from each well. Spectral principal components (PCs) were computed for all wells at each time point and those explaining at least 90% of the variance were calculated. For each well, overlap of the PCs with the positive growth and negative growth controls was calculated and used to determine whether a given well showed bacterial growth. Determining the lowest concentration for each antibiotic that inhibited growth based on these spectral overlaps determined the MIC for each antibiotic used.

Additionally, this example used amalgamation of three wavelength ranges centered at 630 nm, 525 nm, and 475 nm corresponding to red, green, and blue channels respectively to perform ASTs, and termed as a RGB channel assay. In this case, which can be considered an RGB channel assay, two principal components were used which capture >99% of variance. A minimum overlap of 20% with respect to positive control wells was defined as bacterial resistance to the given antibiotic concentration and to extract MICs.

FIG. 4 shows an example of Escherichia coli assay in the reader. The antibiotics concentrations are as in the plate layout and growth is measures as absorption at each excitation wavelength compares with the positive controls. Dark red indicates growth while bright red indicates no growth.

TABLE 1
MIC results corresponding to the assay shown in FIG. 4.

| | MIC | MIC | MIC | MIC |
|---|---|---|---|---|
| | (6 hours) | (8 hours) | (10 hours) | (18 hours) |
| Antibiotics | (μg/mL) | (μg/mL) | (μg/mL) | (μg/mL) |
| Ceftazidime | 0.0625 | 0.0625 | 0.0625 | 0.0625 |
| Meropenem | <=0.03125 | <=0.03125 | <=0.03125 | <=0.03125 |
| Tobramycin | 1 | 1 | 1 | 1 |
| Levofloxacin | <=0.03125 | <=0.03125 | <=0.03125 | <=0.03125 |
| Cefepime | <=0.03125 | <=0.03125 | <=0.03125 | <=0.03125 |
| Gentamicin | 1 | 1 | 1 | 1 |
| Amikacin | 2 | 2 | 2 | 2 |

Instead of using the entire spectral range, measured extinction (absorption) at only a reduced number of wavelength ranges/wavelength range ranges is used, as would be the case for filtered detection with a continuous excitation source or for more discrete excitation sources such as light emitting diode-based excitation with either filtered or broad-band spectral intensity detection. Whole spectra plate reader-determined MICs and those determined from response at only selected wavelength ranges/wavelength range ranges (using from 1 to 7 different wavelength ranges for analysis) were compared against the standard BMD. While even 1 or two wavelength ranges produced reasonable MICs with proper data processing and analysis, three wavelength ranges are used to simulate red/green/blue LED excitation. The categorical and essential agreements of each antibiotic at different time points were compared to BMD. The categorical agreement (CA) was differentiated into susceptible(S), intermediate (I), and resistant (R) categories according to the clinical laboratory and standards institute (CLSI) breakpoints for each bacteria-antibiotic pair used. Based on the agreements with BMD, minor error (mE), major error (ME) and very major error (VME) rates of the plate reader and three-wavelength range assays were evaluated. Minor errors are defined as either new or standard method showing intermediate response with the other being either R or S. Major errors correspond to false resistance classifications of the new method (compared to gold standard BMD), while VMEs are false susceptibilities. Essential agreement (EA) of plate reader and RGB channels were calculated as agreement within two-fold antibiotic concentrations with respect to BMD.

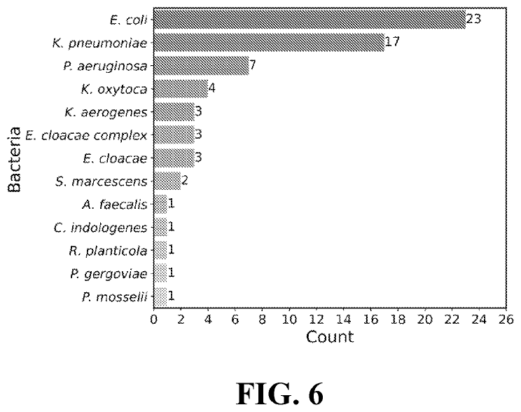

As seen in FIG. 6, 67 Gram-negative positive blood culture samples were analyzed directly via 500×-fold dilution. These samples consist of 13 different bacterial species belonging to 5 different bacterial families i.e., Enterobacteriaceae, Pseudomonadaceae, Yersiniaceae, Alcaligenaceae, and Weeksellaceae which consists of ˜82%, 12%, 3%, 1.5% and 1.5% of the samples respectively.

The resulting MICs were used to compare the average categorial agreement (CA) and average essential agreement (EA) for multiple timesteps with respect to a single endpoint MICs result from BMD (˜18 h).

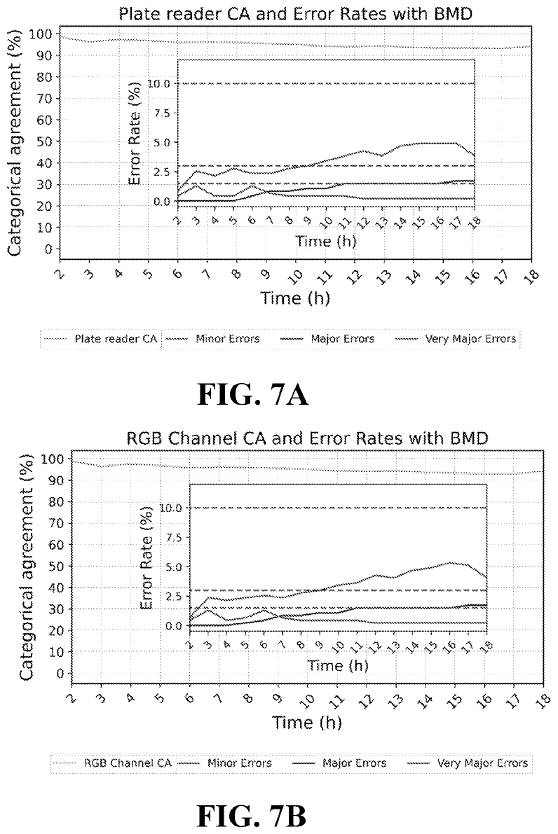

As seen in FIGS. 7A-7B, it is evident that both the plate reader (FIG. 7A) and selected wavelength ranges RGB channel (FIG. 7B) yield >90% categorical agreement with mE, ME and VME each below their respective thresholds of 10%, 3% and 1.5%.

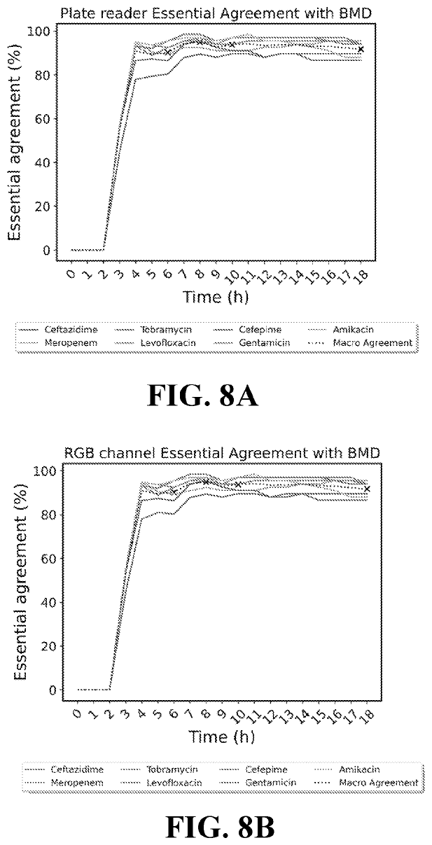

Furthermore, as seen in FIGS. 8A-8B, the average essential agreement of both the plate reader (FIG. 8A), and the RGB channel (FIG. 8B) were calculated using commercial Vitek 2 antibiotic concentration ranges and compared with BMD single endpoint MICs.

FIGS. 8A-8B show average EA corresponding to each antibiotic for 67 BSI Gram negative bacterial isolates. The macro agreements i.e., the average of seven antibiotics show that the EA exceeds 90% after 6 hours. The average essential agreements at 6 hours, 8 hours, 10 hours, and 18 hours were 90%, 95%, 94% and 92%, respectively for both full-spectrum analysis and just the three-channel (RGB) analysis.

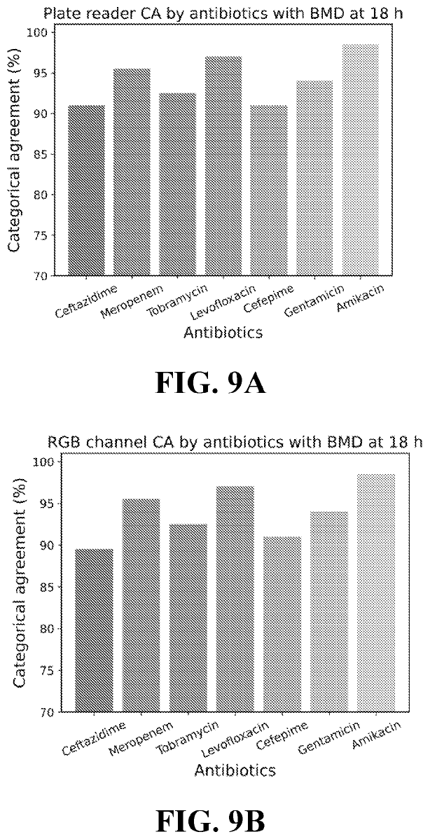

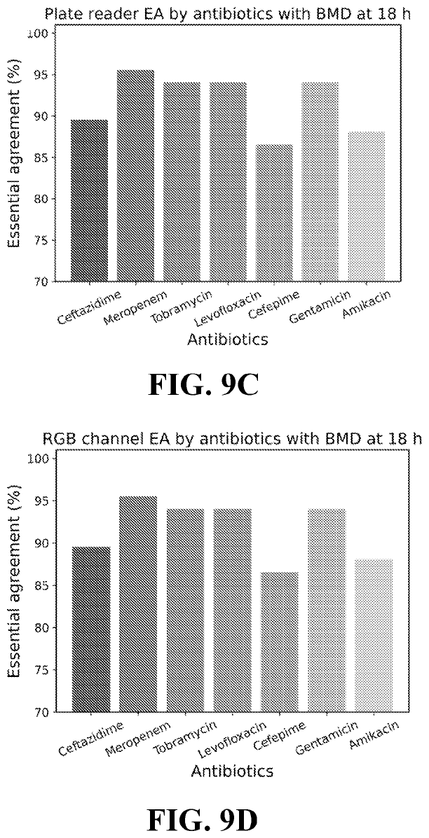

Additionally, the end-point results (˜18 hours) of 500×-fold diluted ASTs results directly from positive blood culture are compared with the standard BMD results (˜18 h). FIGS. 9A-9B show the average CA of seven antibiotics and 67 samples at 18 hours for the full-spectrum plate reader data and RGB channel data, respectively. The average CA for all antibiotics for both the full-spectrum and RGB assays are >90% at 18 hours. Similarly, the average EA at 18 hours for the combination of seven antibiotics and 67 samples is 92% for both full-spectrum, as seen in FIG. 9C, and RGB channel analyses, as seen in FIG. 9D.

Both direct from positive blood culture full-spectrum and RGB analyses give CA exceeding 90% with average error rates (mE, ME and VME) each below the recommended thresholds. The average EA is also ≥90% 6 h onwards for both the assays. This suggests that both the full spectrum optical density and RGB optical density each produce highly accurate phenotypic results directly from positive blood cultures, without addition of contrast agent.

Other colorimetric rapid susceptibility tests can require addition of blood or other dyes that sense a change in oxygen/CO₂ equilibrium through a color change to indicate bacterial growth under antibiotic challenge. This example does not require a contrast agent as either the natural diluted blood culture can work on its own, although in this case as dilution makes this very weak and noisy, but by looking at the baseline in the spectra collected, this example can actually directly use the scattering background (turbidity) that lies beneath the residual color from blood products. Thus, no addition of contrast agent is needed to determine MICs/perform ASTs, and accuracy is improved compared to when examples in which contrast agent is added. This means that scattered light can be used for detection. This makes the assays easier to perform. Further, this example provides instrumentation as some plate readers will not allow stoppage of the experiment in the middle or analyzed data in real time. Thus, there is provided in this example a plate reader with a PCB (printed circuit board) for RGB LED illumination of each well and a PCB for detection, giving an overall flexible and inexpensive instrument.

This example has different capabilities compared to methods relying on analyzing the characteristic oxy-hemoglobin to deoxy-hemoglobin spectral changes as bacteria grow. Accuracy from directly diluted blood culture without addition of contrast agent is increased from approximately ˜70% to approximately 90% or greater. This example also eliminates problems that arise from different amounts of blood being injected into different blood culture tubes, so the intensity/ability to detect peaks varies drastically from blood culture to blood culture, and no additional contrast agent (blood) is added.

Scattered light can be due to bacterial growth. When using that component instead of or in addition to the blood absorptions, the present Categorical Accuracy is improved.

In essence, this example can use either the entire spectrum or just the baseline, or just the blood absorptions (after subtracting off the baseline). It can be advantageous to just use the entire raw spectrum, or the intensities in the R, G, and B wavelength ranges of the raw spectrum. The assay can also use the baseline (turbidity) or the baseline-subtracted.

In summary, a procedure for executing this example is as follows: prepare plates with no contrast agent, add the blood culture to be assayed such that the final dilution is between approximately 500×-1000×, cover the microtiter plate and put in incubating microtiter plate reader, wait for plate reader to finish, analyze spectra, comparing each well to positive and negative control wells-again simple principal components space overlap or separate populations based on a support vector machine-based machine learning model in principal component space to determine growth vs. not.

Thus, it is much easier to pre-prepare plates and is much cheaper as human blood can be rather expensive, depending on source, and has storage and shelf-life issues. Notably, this should no longer depend on oxygen (oxy-deoxy Hb equilibrium) and thus should be directly applicable to anaerobes and yeast as well.

These examples also include a simple and cheap microplate reader. These are printed circuit boards (PCBs) using 96 LEDs and 96 detectors, one each for each well. One example can include one detector overall and one set of LEDs per well. The entire spectrum can be used or turbidity can be more selectively measured by parsing out the background/baseline spectrum and comparing with that from the positive vs. negative controls. The RGB LED wavelength ranges are split out from the plate reader full spectra and only use those intensities for analysis (simulating a plate reader that only collects three intensity measurements, that at the red, green, and blue wavelength ranges).

A high throughput AST with minimal sample preparation and handling will minimize the susceptibility timelines, directly improve patient outcomes, and suppress the alarming rate of antibiotic resistance infections.

### Example 2

In this example, full color images from a camera are recorded and time stamped approximately every 15 minutes. Image and data acquisition is performed using scripts written in python. Incubators were held at 37° C. for bacterial growth. Color changes in all wells were recorded in time. Both individual images and the entire image stacks were analyzed visually to determine MICs. The MIC was determined as the lowest antibiotic concentration at which the color did not change significantly from its initial bright red (or blue, if using anthocyanin) color. Automatic MIC detection was performed in python by automatically locating the position and pixels corresponding to each well, using the red, green, and blue component values for each well to determine the principal components, and comparing the overlap of each well with that of positive growth and negative growth control wells. A variety of machine learning approaches can be used for classification and discrimination of positive vs. negative growth, including support vector machines, neural networks, random forest, and many other generative and discriminative classifiers.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A system comprising:
containers, each container configured to contain at least a portion of a biological sample and an antimicrobial agent;
a light source disposed on a first side of the containers and configured to shine light onto the containers;
a detector configured to detect biological sample growth using at least one of turbidity, one or more colorimetric contrast agents, or one or more endogenous chromophores;
one or more processors; and
a memory storing instructions thereon that, when executed by one or more of the processors, cause one or more of the processors to:
capture, with the detector, a susceptibility profile of at least one of the containers; and
determine, from the susceptibility profile, an effectiveness of the antimicrobial agent in at least one of the containers.

2. The system of claim 1, wherein:
each of the containers are configured to contain at least one of a varying concentration of the same antimicrobial agent or a different type of antimicrobial agent;
the susceptibility profile is an intensity profile; and
the instructions further cause one or more of the processors to:
determine, from the intensity profile, a first intensity of the light at a first wavelength range; and
compare the first intensity of the light to a control;
wherein the effectiveness of the antimicrobial agent is a minimum inhibitory concentration (MIC) of the antimicrobial agent; and
wherein the MIC of the antimicrobial agent in at least one of the containers is based upon the comparing.

3. The system of claim 2, wherein the instructions further cause one or more of the processors to:
determine, from the intensity profile, a second intensity of the light at a second wavelength range;
determine, from the intensity profile, a third intensity of the light at a third wavelength range;
compare the second intensity of the light to the control; and
compare the third intensity of the light to the control.

4. (canceled)

5. The system of claim 3, wherein the light source comprises three constituent components at the first, second, and third wavelength ranges.

6. The system of claim 5, wherein the first, second, and third wavelength ranges correspond to red, green, and blue light wavelength ranges respectively.

7. The system of claim 6 further comprising:
additional detectors;
wherein each detector is aligned with a respective container.

8. The system of claim 7, wherein the light source comprises light emitting diodes (LEDs);
wherein each LED is aligned with a respective container and opposite a respective detector.

9. The system of claim 8 further comprising:
an incubator configured to contain the containers.

10. The system of claim 9 further comprising:
a transparent cover film covering the containers;
wherein the instructions further cause one or more of the processors to maintain a temperature gradient in the incubator such that condensation does not form on the transparent cover film.

11. The system of claim 10, wherein the containers comprises a well plate.

12.-15. (canceled)

16. A system for testing antimicrobial susceptibility comprising:
an incubator;
a well plate comprising wells, the wells containing biological samples and an antimicrobial agent;
a respective light source for each of the wells, each light source disposed in the incubator and configured to shine light onto the wells;
a transparent cover film covering the well plate; and
a respective detector for each of the wells, each detector configured to capture an intensity profile of the well plate disposed between the respective detector and the respective light source.

17. (canceled)

18. The system of claim 16, wherein each light source comprises a light emitting diode (LED), each LED configured to emit light at three distinct wavelength ranges; and
wherein each detector is configured to detect a respective intensity of each of the three wavelength ranges, the intensity indicative of an effectiveness of the antimicrobial agent.

19. (canceled)

20. The system of claim 16, wherein the incubator is configured to maintain a temperature gradient maintain a temperature gradient in the incubator such that condensation does not form on the transparent cover film.

21. The system of claim 1 further comprising:
an incubator configured to contain the containers; and
a cover film covering the containers;
wherein:
each of the containers are configured to contain at least one of a varying concentration of the same antimicrobial agent or a different type of antimicrobial agent;
the effectiveness of the antimicrobial agent is a minimum inhibitory concentration (MIC) of the antimicrobial agent;
the system is configured for rapid MIC determination direct from the biological sample;
the susceptibility profile comprises a colorimetric readout of bacterial growth in the presence of the antibiotic in the container;
the rapid MIC determination is from 4-7 hours;
a time duration between a preincubation step of the biological sample and the rapid MIC determination is less than 17 hours.

22. An automated minimum inhibitory concentration (MIC) determination process comprising:
incubating containers containing a biological sample and a concentration of an antimicrobial agent;
exposing the containers to a light;
detecting colorimetric growth of the biological sample in the containers; and
determining, based on the detecting, the MIC of the concentration of the antimicrobial agent.
